(19) United States
(12) Patent Application Publication
(10) Pub. No.: US 2006/0172982 A1
(43) Pub. Date: Aug. 3, 2006

(54) Formulation comprising tetracycline or derivative and method of treating or preventing infections and inflammatory conditions

(76) Inventor: Wallace J. Gardner, Cambridge, MA (US)

Correspondence Address: NIELDS & LEMACK, 176 EAST MAIN STREET, SUITE 7, WESTBORO, MA, 01581, US

(21) Appl. No.: 11/046,467

(22) Filed: Jan. 28, 2005

(51) Int. Cl. A61K 31/65

(52) U.S. Cl. 514/152

(57) ABSTRACT

Therapeutic composition and method for treating or preventing various conditions and diseases, including inflammatory conditions, allergies, particularly seasonal allergies, and the common cold. In a preferred embodiment, the therapeutic composition is a formulation comprising an antibiotic, preferably a tetracycline, most preferably doxycycline, which has not been chemically modified to eliminate antimicrobial efficacy. The antibiotic is preferably in a vehicle to form a salve or paste for topical application to the skin or the nasal cavity. The therapeutic composition most preferably is a self-applied formulation that consists essentially of a tetracycline, most preferably doxycycline, which has not been chemically modified to eliminate antimicrobial efficacy, and a vehicle such as boric acid, zinc oxide or petroleum jelly.

### BACKGROUND OF THE INVENTION

Many common diseases and conditions, including inflammatory diseases and conditions, such as the common cold, allergies, sinusitis, diseases of the gastrointestinal tract (including those that manifest themselves in stomach and bowel problems), influenza, halitosis, pneumonia, shingles etc., may be caused by viruses and/or microorganisms. Often the source of the microorganisms and viruses is the sinuses, typically via the oral cavity, especially the ear, nose and throat passages. Once the microorganisms and/or viruses are resident in the oral cavities or sinuses (e.g., the maxillary, frontal, ethmoid and ophenoidal), they can continually cause inflammation and infection through circulation in the blood stream. Continual reduction or elimination of these microorganisms and viruses would reduce chronic infection in the body.

Conventional treatments for the common cold and seasonal allergies usually involve oral and nasal decongestants that relieve common systems. Unfortunately, they are often less effective than desired, and have various side effects, including sedation, dry mouth, dry eyes, etc.

Inflammatory skin conditions such as psoriasis, which is an autoimmune skin disease, are experienced by an estimated 5-6 million Americans and about 80 million people around the world. Although not life threatening, psoriasis can cause considerable discomfort to sufferers, and is often unsightly. Typical locations affected most often are the elbows, knees, scalp and back. Common systems include itchiness, burning, dry skin and blistering. Psoriatic arthritis also can result. The etiology of psoriasis has not been fully elucidated. It generally appears as patches of raised red skin covered by a flaky white buildup. In certain kinds of psoriasis, it also has a pimple-ish (pustular psoriasis) or burned (erythrodermic) appearance. Cortisone creams or ointments are often used for treatment. However, if only cortisone creams are used for treatment, the psoriasis can become resistant and may recur if use of the creams is terminated. Anthralin is another common topical treatment, as is a combination of a variety of topical drugs if the psoriasis is not responsive to one treatment or the other. Ultraviolet light, either natural or artificial, also can be effective.

Although more severe cases of psoriasis are treated systemically, often deleterious side effects are involved.

Shingles is caused by the varicella-zoster virus, the same virus that causes chickenpox. The virus is usually dormant in the nerve tissue once an individual contracts chicken pox. The virus can reappear in the form of shingles later in life. Shingles is estimated to affect 2 in every 10 persons in their lifetime.

It therefore would be desirable to provide a therapeutic composition and kit that effectively prevents and/or treats the common cold, allergies and pneumonia.

It further would be desirable to provide a therapeutic composition and method for treating or preventing inflammatory skin conditions and lesions such as psoriasis, acne and shingles.

It further would be desirable to provide a composition and method for reducing or eliminating the plaque caused by psoriasis and the lesions caused by shingles.

### SUMMARY OF THE INVENTION

The problems of the prior art have been overcome by the present invention, which provides a therapeutic composition and method for treating or preventing various conditions and diseases, including inflammatory conditions, allergies, particularly seasonal allergies, and the common cold. In a preferred embodiment, the therapeutic composition is a formulation comprising an antibiotic, preferably a tetracycline, most preferably doxycycline, and preferably which has not been chemically modified to eliminate antimicrobial efficacy. The antibiotic is preferably in a vehicle to form a salve or paste for topical application to the skin or the nasal cavity. The therapeutic composition most preferably is a self-applied formulation that consists essentially of a tetracycline, most preferably doxycycline, which has not been chemically modified to eliminate antimicrobial efficacy, and a vehicle such as boric acid, zinc oxide or petroleum jelly.

A further embodiment of the present invention relates to a formulation that includes a kit comprising one or more of the therapeutic compositions mentioned above along with instructions for use in treating or preventing various conditions or diseases.

The present invention also relates to a method of treating or preventing the common cold and allergies, by applying a salve of the present invention directly to the nasal cavity.

### DETAILED DESCRIPTION OF THE INVENTION

The therapeutic compositions of the present invention are topical delivery compositions that have anti-inflammatory activity, and comprise an antibiotic, preferably a tetracycline that optionally has not been chemically modified to eliminate antimicrobial efficacy, most preferably doxycycline. The therapeutic compositions of the present invention can be readily applied directly to or in the vicinity of the nasal cavity, most preferably to line the nasal cavity, to treat or prevent infections and viruses, including those responsible for the common cold, and also to treat or prevent allergies, particularly chronic and seasonal allergies, such as those due to hypersensitivity to pollen and/or mold, including but not limited to allergic rhinitis or hay fever. The therapeutic compositions of the invention reduce or eliminate symptoms typical of the common cold and allergies, including runny nose, itchy eyes, skin or mouth, watery eyes, sneezing, bronchial mucus congestion (both in the nose and in the chest), and coughing. Application to the nasal passages has decongesting activity, and improves breathing. In addition, the therapeutic compositions of the present invention facilitate the prevention, treatment and management of inflammatory skin conditions, including seborrheic dermatitis, psoriasis, focciculitis, rosacea, perioral dermatitis, acne, impetigo, shingles, etc.

Suitable tetracyclines useful in the compositions of the present invention include apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, senociclin, tetracycline, and combinations of the foregoing. Doxycycline is particularly preferred.

The therapeutic compositions of the present invention also include a carrier that is a compatible, nontoxic substance suitable to deliver the antibiotic topically or in the nasal cavity. Preferred carriers include petroleum jelly (e.g., Vasoline®), zinc oxide, boric acid (preferably boric acid ointment), and mixtures thereof. Additional agents, such as vitamins A, C and/or E, optionally may be added to the formulation. The combination of the carrier, the antibiotic and any optional ingredients form salves or pastes. The active compound can be mixed, preferably under sterile conditions, with the carrier, and optionally with preservatives, stabilizing agents, emulsifying agents, dispersing agents, suspending agents, thickening agents, coloring agents, perfumes, excipients and the like.

Inactive ingredients can be incorporated into the formulations to improve cosmetic acceptability. Examples of such ingredients are emulsifiers, humectants, surfactants, preservatives, fragrances, coloring agents, emollients, and fillers.

The amount of antibiotic used in the formulation depends upon the frequency of the dose, the particular infection or malady to be treated or prevented, the severity of the infection or malady being treated or prevented, the method of local administration of the composition and the effect desired (including whether the treatment is a prophylactic or therapeutic application). Preferably the formulation contains from about 100 to about 500 mgs tetracycline, most preferably doxycycline, per ounce of carrier in the formulation. More preferably the formulation contains from about 100 to about 200 mgs of the antibiotic, preferably doxycycline, per ounce of carrier. The formulation is prepared by mixing the tetracycline with the carrier, prior to, during or after the optional addition of inactive ingredients.

In the preferred form of the present invention, the therapeutic composition is a salve or paste and is self-administered topically (which includes application to the nasal cavity). The salve or paste is preferably applied at least once a day, more preferably at least twice a day, such as in the AM and in the PM. Salves with different concentrations of antibiotic can be used depending upon the severity of the condition being treated. As the condition improves, the concentration of antibiotic in the formulation can be decreased.

Therapeutic compositions with different carriers (e.g., petroleum jelly, boric acid ointment and zinc oxide ointment) can be used in sequence. For example, depending upon the results obtained and the severity of the malady being treated, the petroleum jelly salve can be applied initially. If the malady being treated does not respond adequately to this treatment, or becomes resistant thereto, the boric acid ointment can be applied. Similarly, if the malady being treated does not respond adequately to this treatment, or becomes resistant thereto, the zinc oxide ointment can be applied. The order of application can be varied.

The terms “treatment” or “treating” or “treat(s)” as used herein in reference to conditions or diseases include 1) preventing such conditions or diseases from occurring in a subject who may be predisposed to such condition or disease or diseases but who has not yet been diagnosed as having it or them; 2) inhibiting these conditions or diseases, i.e. arresting their development; or 3) ameliorating or relieving the symptoms of these condition or diseases, i.e., causing regression of the disease state. “Therapeutic” as used herein includes compositions for prophylactic treatment of conditions or diseases.

The amount of antibiotic, e.g. doxycycline, used in the therapeutic compositions and the dosage selection is an amount and dosage effective to achieve anti-inflammatory activity, i.e. the amount necessary to effectively treat conditions such as the common cold, allergies and skin disorders such as psoriasis and shingles. Although suitable amounts of antibiotic in the therapeutic composition are as discussed above, those skilled in the art will determine optimum concentrations and dosages from clinical experience in order to carry out the method of the invention.

In one embodiment of the present invention, the composition applied to the skin is maintained in a covered state, minimizing exposure to air, to minimize or prevent it from drying or being wiped off the applied area. The covering material is not particularly limited. Suitable covering materials include cotton, gauze and conventional patch material. The material can be a non-breathable layer such as sheets of plastic, polyethylene, polyvinyl chloride, wax paper, foil, latex, etc., and combinations thereof. Those skilled in the art will recognize that any non-breathable substance (defined as a substance that does not allow the exchange of gases through its membrane) that is not deleterious to the active ingredient being used and that does not cause any irritation upon contact with skin can be used.

If desired, the patch material can be secured to the skin by any suitable means, such as with a bandage having adhesive or fasteners. Compression also can be used. The material can be conveniently designed to accommodate specific topical sites. For example, it can be shaped in the form of a glove to be worn on one hand of the individual, or can be shaped in the form of a band to be worn on the arm or leg of the individual.

The salves of the present invention also can be absorbed into a substrate such as fabric or cloth and applied to the site of application using the substrate. The substrate can remain on the site to minimize or eliminate the salve from drying out and to prevent it from rubbing off due to contact with clothing, etc.

In the embodiment where the salve is applied directly to the nasal passages, the amount of salve applied is not particularly limited. Preferably salve is applied inside each nostril to line the nose and/or around the outer edge of each nostril. The actual amount used should depend upon the size of the nostril and should not prevent the individual being treated from breathing through the nostrils. After application, preferably the patient gently blows the nose to remove excess salve.

Application directly to the nasal passages is particularly effective for preventing symptoms of the common cold and allergies during air travel and in environments where the air is dry.

### EXAMPLE 1

The contents of two 100 mg capsules of doxycycline were added to one ounce of petrolatum and mixed thoroughly to disperse the doxycycline uniformly in the petrolatum carrier to form a salve.

A patient with a finger covered with psoriasis scabs applied the salve applied the salve liberally to all infected areas, and was instructed to keep the finger coated at all times, applying additional salve as needed. The scabs were healed within 48 hours.

### EXAMPLE 2

The salve prepared as in Example 1 was liberally applied to the face of a 91 year-old patient with red fungal-like sores or lesions on his face. The salve was re-applied as needed to keep the lesions coated. The lesions healed within about one week. The patient continues to use the salve prophylactically, and the lesions have not returned.

### EXAMPLE 3

A patient with pollen allergies placed a salve prepared as in Example 1 in each nostril daily by lining each nostril followed by gently blowing the nose to remove excess and allow more air to reach the lungs. Allergy symptoms of runny nose and watery eyes were relieved.

### EXAMPLE 4

Salves as prepared in Example 1 were used to line the nasal cavities of various individuals prior to and during airplane travel. All reported no or minimal dryness, irritation and congestion, and ease of breathing. None contracted the common cold.

### EXAMPLE 5

The contents of two 100 mg capsules of doxycycline were added to one ounce of boric acid ointment and mixed thoroughly to disperse the doxycycline uniformly in the boric acid ointment carrier to form a salve.

A patient with seasonal allergies placed the salve in each nostril daily by lining each nostril followed by gently blowing the nose to remove excess and allow more air to reach the lungs. Allergy symptoms of runny nose and watery eyes were relieved.

What is claimed is:

1. A therapeutic composition having anti-inflammatory activity comprising an amount of a tetracycline effective for treating an inflammatory condition, said tetracycline being dispersed in a salve selected from the group consisting of petroleum jelly, zinc oxide and boric acid, said therapeutic composition being in topical delivery form and adapted for self-administration.

2. The therapeutic composition of claim 1, wherein said tetracycline has not been chemically modified to eliminate antimicrobial efficacy.

3. The therapeutic composition of claim 1, wherein said therapeutic composition is devoid of a morpholinoamino alcohol or pharmaceutically acceptable salt thereof.

4. The composition of claim 1, wherein said tetracycline is doxycycline.

5. The composition of claim 1, wherein said anti-inflammatory activity is anti-psoriatic activity.

6. A method of treating or preventing psoriasis in a patient comprising topically applying to the skin of said patient affected with psoriasis the therapeutic composition of claim 1.

7. The method of claim 9, further comprising covering said applied composition.

8. The method of claim 10, wherein said tetracycline is doxycycline.

9. A kit, comprising a tetracycline, a salve as a carrier for said tetracycline, and instructions for use of said tetracycline and carrier for treating symptoms of the common cold or allergies.

10. The kit of claim 12 wherein said tetracycline is doxycycline.

11. The kit of claim 12, wherein said instructions for use require application of said salve to the nasal cavity.

12. A method of relieving symptoms in a patient due to the common cold or allergies, comprising directly applying to the nasal passages of said patient an effective amount of a salve comprising tetracycline dispersed in a carrier selected from the group consisting of petroleum jelly, boric acid and zinc oxide.

13. The method of claim 12 wherein said tetracycline is doxycycline.

14. The method of claim 12, further comprising blowing the nose of said patient to expel excess salve.
